(19) United States
(12) Patent Application Publication
(10) Pub. No.: US 2003/0045832 A1
(43) Pub. Date: Mar. 6, 2003

(54) Endotracheal tube

(76) Inventors: Jorje I. Bassuk, Coconut Grove, FL (US); Jose A. Adams, Miami, FL (US)

Correspondence Address: MCHALE & SLAVIN, 4440 PGA BLVD, SUITE 402, PALM BEACH GARDENS, FL, 33410

(21) Appl. No.: 09/945,916

(22) Filed: Sep. 4, 2001

(51) Int. Cl. A61M 29/00

(52) U.S. Cl. 604/103.03; 604/910

(57) ABSTRACT

An improved endotracheal tube has a balloon at the distal end. The balloon is flexibly attached to the tube by a connector sleeve. The connector sleeve includes an area that is unattached to either the balloon or the tube. The unattached area of the connector sleeve permits relative movement between the balloon and the tube.

### FIELD OF THE INVENTION

This invention relates to balloon catheters, specifically for use as endotracheal tubes, with an improved connection between the catheter and the balloon.

### BACKGROUND OF THE INVENTION

Balloon catheters are used to create a blockage in a hollow space or tubular body structure, in animals, either to hold the catheter in place or to prevent reflux past the catheter tip or both. The bore in the catheter shaft is used for administering treating agents or for suction of the space beyond the balloon or both.

The trachea is a cartilaginous tube for carrying air through the throat from the nose and mouth to the bronchi of the lungs. The tracheal mucosae is a delicate layer of cells lining the trachea.

Endotracheal tubes are used to provide a clear passageway through the trachea between the lungs and the atmosphere. Endotracheal tubes are put in place for various reasons, such as bypassing a damaged area of the throat which is causing an obstruction to breathing, providing a clear passageway for inhalation and exhalation, providing a connection to a ventilator, and others.

In most instances, the endotracheal tube has a circumferential balloon connected about the distal end which is inflated after the tube is positioned in the trachea. The balloon may be inflated with any bio-compatible liquid or gas. The balloon may be a high volume-low pressure cuff or a high pressure-low volume cuff. The balloon may be made of elastic material which stretches upon inflation or pliable material which merely unfolds. Usually, the inflated balloon serves to secure the tube against movement within the trachea. This requires a certain amount of pressure to be exerted by the balloon against the tracheal mucosae. Any inadvertent or intentional movement of the tube, after the balloon has been inflated, can cause injury to the mucosae through frictional forces or puncture by the tip of the tube.

Intubated patients suffer injuries when the proximal end of the endotracheal tube is displaced causing movement of the distal end and balloon. Typically, such action is the result of moving the patient or of moving the equipment connected to the proximal end of the tube. For example, a hand held breathing bag may be connected to the proximal end of the tube and the operator may push, longitudinally, on the bag and tube when squeezing to provide positive inhalation.

### DESCRIPTION OF THE PRIOR ART

There are balloon catheter and endotracheal tube patents, too numerous to mention. One example, is U.S. Pat. No. 4,793,359 issued Dec. 27, 1988 to Sharrow. The patent teaches an angioplasty balloon catheter placed in an artery to position a laser carried in the bore of the catheter. The elongated balloon is circumferential to the distal end of the shaft of the catheter with the distal tip of the catheter extending beyond the balloon. The forward end and the rearward end of the balloon are attached to the shaft of the catheter. When the balloon is inflated the forward end wall is perpendicular to the shaft of the catheter centering the laser in the artery. After inflation, the balloon position is fixed in relation to the catheter.

U.S. Pat. No. 6,264,631 B1 issued Jul. 24, 2001 to Willis et al teaches a balloon catheter with a balloon that extends beyond the distal tip of the catheter shaft upon inflation to protect tissues from contact with the catheter tip. The balloon is constructed to force inflation medium into the forward end of the balloon distending it beyond the catheter tip. After inflation, the balloon position is fixed in relation to the catheter.

What is needed in the art is a balloon catheter that, when inflated, protects delicate tissues from frictional or contact damage in which the balloon extends beyond the catheter tip and is longitudinally and radially movable in relation to the catheter after inflation.

### SUMMARY OF THE INVENTION

A balloon catheter is disclosed having an elongated tubular walled shaft having a central bore with a proximal end and a distal end. An inflatable balloon surrounds the distal end. The balloon has an inflation opening communicating with the interior of the balloon. An inflation tube is connected to the tubular walled shaft extending from the proximal end of the catheter to the balloon and connected to the inflation opening for providing inflation to the balloon. A flexible connection between the balloon and the tubular shaft is available for attaching the balloon to the distal end of the shaft. The connection means having an unattached length greater than the distance between the balloon and the shaft whereby the tubular shaft and the balloon are relatively movable.

An objective of the instant invention to teach an endotrachael tube having a balloon about the distal end that is movable longitudinally and radially with respect to the tube to protect surrounding tissue from frictional and contact injury.

It is a further objective of the instant invention to teach an endotrachael tube which includes an inflation tube connected to the interior of the attached balloon.

It is yet another objective of the instant invention to teach a flexible connection sleeve between the balloon and the distal end of the tube.

It is a still further objective of the invention teach a movable balloon that always extends beyond the distal tip of the tube when inflated.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### DESCRIPTION OF THE FIGURES

FIG. 1 shows a longitudinal cross section of the endotrachael tube of this invention with the distal end of the tube forwardly displaced;

FIG. 2 shows a longitudinal cross section of the endotracheal tube with a modified connection means and the distal end of the tube rearwardly displaced; and

FIG. 3 is a longitudinal cross section of the endotracheal tube with a modified connection means; and

FIG. 4 is a transverse cross section along line 4-4 of FIG. 1.

### DETAILED DESCRIPTION OF THE INVENTION

The endotracheal tube 10, shown in FIGS. 1, 2 and 3, is formed with an elongated walled shaft 11 containing a bore 12 having open proximal and distal ends 13 and 14, respectively. The proximal end 13 may be fitted with Luer-type connectors for use with various medical implements or adapters for implements.

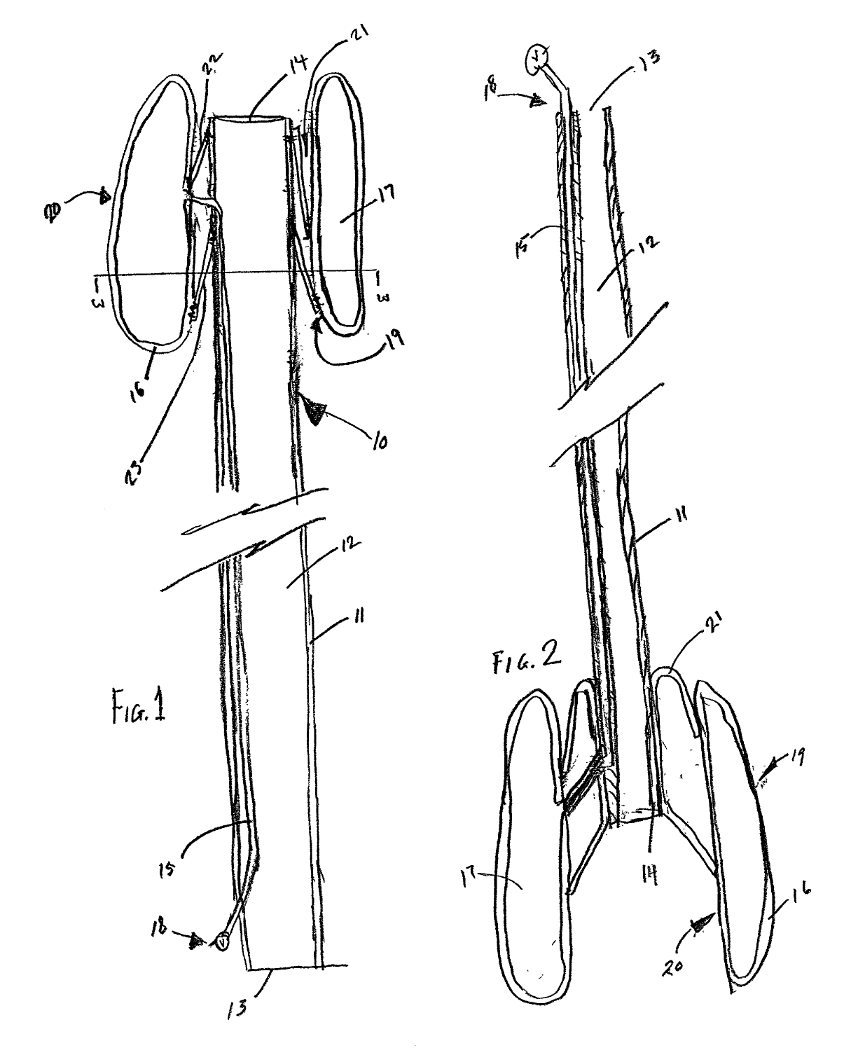

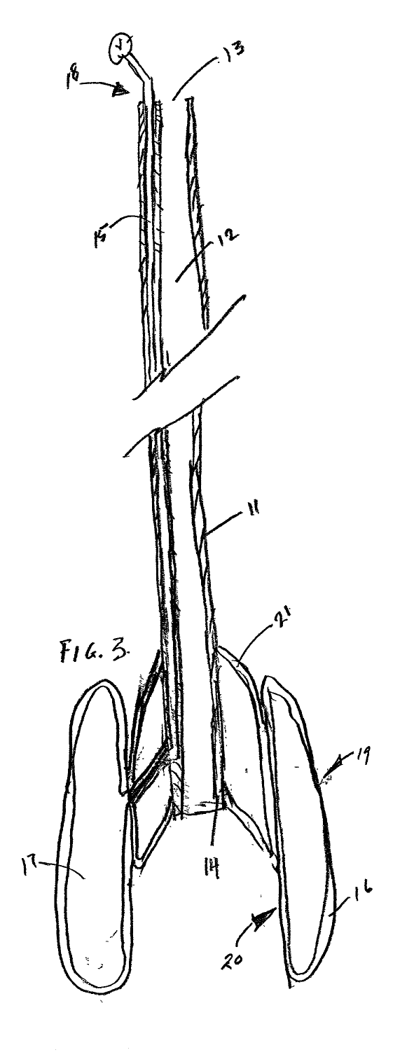

The walled shaft 11 supports an inflation tube 15 which extends from the proximal end to the distal end and is connected to the interior 17 of the balloon 16 by a valved opening 18. The inflation tube 15 may be a separate or integral tube attached to the walled shaft inside the bore 12, as shown in FIG. 1, or on the outside of the shaft. The inflation tube may also be formed as a small diameter bore in the walled shaft 11, as shown in FIG. 2. The inflation tube has a one-way valve 18 through which the inflation medium enters the interior 17 of the balloon. The valve may be located at any point along the length of the inflation tube. For ease in deflating the balloon, the valve can be located at the proximal end of the inflation tube.

The balloon 16 is made of a polymer or rubber that will inflate into an elongated dough-nut shape having a specified inside diameter 19 and outside diameter 20. The inside diameter is greater than the outside diameter of the walled shaft 11 to permit relative movement between the tube and the balloon.

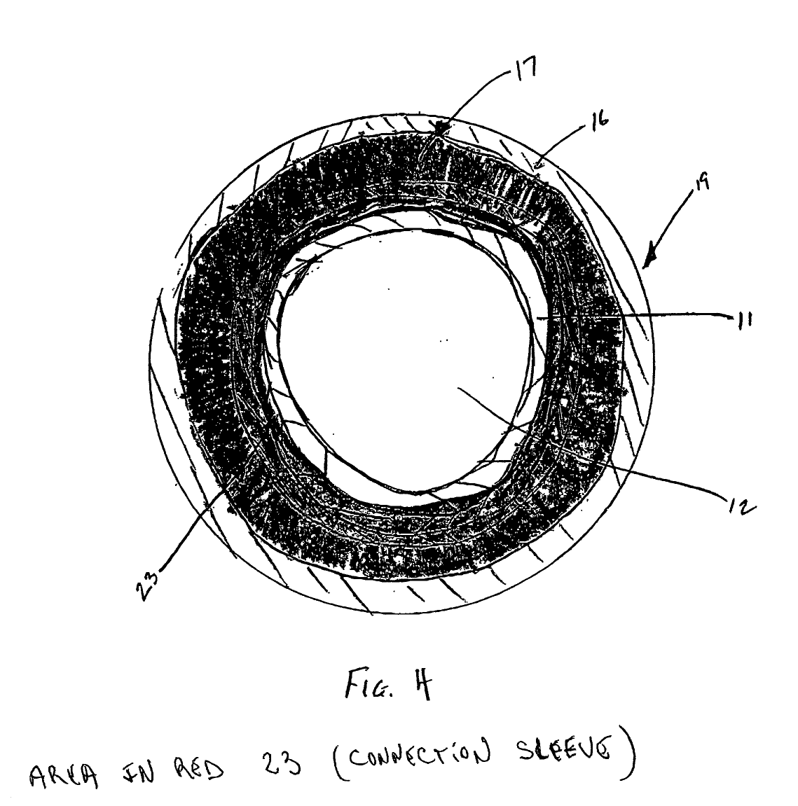

Between the walled shaft 11 and the balloon 16 there is a flexible connection sleeve 21. As shown in FIG. 1, the sleeve is a thin imperforate membrane and formed as a forward disk 22 and a rear disk 23 enclosing the area between the disks. Not shown is a connection sleeve having only one disk centrally attached to the balloon. The outer circumference of the disks is attached to the balloon and the inner circumference is attached to the walled shaft. The connection sleeve may be connected to the shaft and balloon by adhesive, autogenous bonding, molding, etc. The radial distance between the inner and outer circumference of the disks is approximately the same as the distance the balloon can longitudinally travel along the walled shaft 11. Of course, the connection sleeve 21 may take other forms, such as a tubular sleeve having the central area attached to either the walled shaft or to the inside diameter of the balloon with opposite ends free. The edges of the free ends may be attached to either the shaft or the balloon. The length of the sleeve between the central portion and the edges approximates the distance the balloon can move. For example, the central portion of the connector sleeve may be attached to the walled shaft 11 and the free edges may be attached to the inside diameter of the balloon, as shown in FIG. 2, or vice versa, as shown in FIG. 3. This construction allows relative movement between the shaft and the balloon.

To insure that the distal end 13 of the walled shaft 11 does not contact surrounding body tissue, the connector sleeve is attached, as shown in FIGS. 1 and 2, to limit distal movement of the shaft relative to the balloon. In operation, once the balloon has been inflated and makes frictional contact with the surrounding tissue, such as the tracheal mucosae, it should not be moved. The proximal end of the walled shaft 11 or the patient may be jostled and the distal end of the shaft may reciprocate within the balloon to the limit of the connection sleeve without disturbing the balloon.

Because of the flexible connection between the balloon and the tube shaft, some radial movement of the tube, in relation to the inflated balloon, may also be accommodated without movement of the balloon.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification and drawings.

What is claimed is:

1. A balloon catheter comprising an elongated tubular walled shaft having a central bore with a proximal end and a distal end, an inflatable balloon surrounding said distal end, said balloon having an inflation opening communicating with the interior of said balloon, an inflation tube connected to said tubular walled shaft extending from the proximal end of said catheter to said balloon and connected to said inflation opening for providing inflation medium to said interior of said balloon, flexible connection means between said balloon and said tubular shaft for attaching said balloon to said distal end of said shaft, said connection means having an unattached length greater than the distance between said balloon and said shaft whereby said tubular shaft and said balloon are relatively movable.

2. A balloon catheter of claim 1 further comprising said balloon having a forward end and a rear end, said forward end of said balloon being disposed distally of said distal end of said tubular walled shaft.

3. A balloon catheter of claim 2 further comprising said connection means formed as a disk with a central aperture, said disk having an inner circumference about said aperture and an outer circumference about the periphery, said outer circumference attached to said balloon and said inner circumference attached to said tubular walled shaft, the unattached distance between said inner circumference and said outer circumference approximating the distance that said balloon and said tubular walled shaft can move relative to each other.

4. A balloon catheter of claim 2 further comprising said connection means formed as a tubular sleeve with a central portion and free end portions, said central portion concentric to said tubular walled shaft and said free end portions attached to said balloon, the unattached distance between said central portion and said free end portions approximating the distance that said balloon and said tubular walled shaft can move relative to each other.

5. A balloon catheter of claim 2 further comprising said connection means formed as a tubular sleeve with a central portion and free end portions, said central portion attached to said balloon and said free ends attached to said tubular walled shaft, the unattached distance between said central portion and said free end portions approximating the distance that said balloon and said tubular walled shaft can move relative to each other.
